(19) United States
(12) Patent Application Publication
(10) Pub. No.: US 2024/0358654 A1
(43) Pub. Date: Oct. 31, 2024

(54) PREPARATION METHOD AND USE OF POROUS STARCH FOR ENCAPSULATING PROBIOTIC

(71) Applicant: Zhejiang University, Hangzhou (CN)

(72) Inventors: Enbo Xu, Hangzhou (CN); Qingqing Zhu, Hangzhou (CN); Jingsong Feng, Hangzhou (CN); Haibo Pan, Hangzhou (CN); Siyu Yao, Hangzhou (CN); Tian Ding, Hangzhou (CN); Donghong Liu, Hangzhou (CN); Xingqian Ye, Hangzhou (CN)

(21) Appl. No.: 18/641,418

(22) Filed: Apr. 21, 2024

Related U.S. Application Data
(63) Continuation of application No. PCT/CN2023/107924, filed on Jul. 18, 2023.

(30) Foreign Application Priority Data
Apr. 26, 2023 (CN) 202310461438.9
Apr. 26, 2023 (CN) 202310465246.5

(51) Int. Cl. A61K 9/51; A61K 35/00; A61K 35/741

(52) U.S. Cl. CPC A61K 9/5192; A61K 9/5161; A61K 35/741; A61K 2035/115

(57) ABSTRACT

The present disclosure discloses a preparation method of porous starch for encapsulating probiotic, and belongs to the field of food processing. The present disclosure provides porous starch with controllable pore size and morphology by regulating the ratio of amylose to amylopectin (quantitative compounding), keeping starch hydroxyl sites exposed (concentration cultivation), and building interfacial tension to stretch starch chains (convection drying), and uses the porous starch to encapsulate probiotics, which can improve the retention rate of probiotics, reduce the loss of probiotics during food processing and transportation, and thus retain the biological functions of probiotics to a maximum extent.

### CROSS-REFERENCE TO RELATED APPLICATIONS

The present application is a Continuation Application of PCT Application No. PCT/CN2023/107924, filed on Jul. 18, 2023, which claims the priorities of Chinese Application No. 202310465246.5, filed on Apr. 26, 2023, and Chinese Application No. 202310461438.9, filed on Apr. 26, 2023, the entire contents of which are hereby incorporated by reference.

### TECHNICAL FIELD

The present disclosure relates to a preparation method and use of porous starch for encapsulating probiotics, and in particular to a processing method utilizing interfacial tension formed by ethanol evaporation and moisture migration to stretch the amylose and amylopectin with different chain lengths in the system to form porous starch with “perfect circular” pore shape and controllable pore size. In the present disclosure, the porous starch with nanoscale pores is used as a carrier to encapsulate probiotics by using adsorption characteristics of the nanoscale pores of the porous starch. The slightly gelatinized condition promotes the structural change of the porous starch to encapsulate the probiotics, thereby improving the retention rate of the probiotics in complex environments.

### DESCRIPTION OF THE PRIOR ART

Porous starch (PS), as a commonly used modified starch, has higher adsorption efficiency, solubility and swelling capacity than the natural starch, and is widely used as a carrier for biologically active substances, an adsorbent for pollutants and an encapsulating agent for dietary supplements. Traditional methods for preparing PS, such as biological enzymatic method, ethanol-alkali method, acid hydrolysis method and molecular insertion method, often cause large shrinkage and deformation of starch granules. So far, there is still no technology which can control the pore properties, such as pore size, pore volume, porosity and morphology.

Furthermore, probiotics have been shown to be beneficial to gastrointestinal function, which can survive in the mouth and stomach and growth in the intestine, thereby showing beneficial biological effects on host diseases, such as antibiotic-associated diarrhea, necrotizing enterocolitis, intestinal diseases, etc. However, in food processing, probiotics are easily exposed to active environment such as heat, oxygen, and water, which affect their survival.

### SUMMARY

The present disclosure provides a preparation method for PS, which can control the pore size, by combining technical processes such as compounding purified amylose and amylopectin, ultrasonic melting to expose hydroxyl groups, ethanol anti-solvent method, convection drying and stretching to form pores, to inventively connect the process in series such as regulation of the ratio of amylose to amylopectin, promoting dispersed state of starch chains, formation of starch-ethanol V-type complex, and establishment of interfacial tension for stretching, in combination with the negative correlation between starch pore size and the ratio of amylose to amylopectin, to effectively obtain porous starch with pore sizes in the range of 1 to 1000 nm.

The present disclosure adopts the following technical solution: a preparation method for porous starch for encapsulating probiotics, including: using an ultrasonic physical field to promote the gelatinization and melting of the compound starch solutions with different ratios of amylose to amylopectin, performing alcohol precipitation on the gelatinized solutions of starch chains in dispersed state timely, and slowly blast drying the agglomerated starch chains to obtain porous starch with pore sizes in the range of 1 to 1000 nm and “perfect circular” pore shape. Specifically, performing ultrasonic treatment on the compound starch suspensions, and then performing alcohol precipitation on the ultrasonic treated solutions; after performing concentration cultivation on the ultrasonic treated solutions precipitated by alcohol, performing solid liquid separation, and drying the precipitates to obtain the porous starches with controllable multi-level pore sizes and morphology. The compound starch is composed of amylose and amylopectin, with amylose accounting for 50 to 80% of the compound starch.

In the present disclosure, the prepared amylose-amylopectin suspension is subjected to ultrasonic melting, which promotes the starch chains to maintain a uniformly dispersed state in the solution and gives the polar molecules of ethanol a good chance to enter the cavities of the spiral starch chains (that is, the establishment of the anti-solvent method). After the quantitatively introduced ethanol polar molecules are efficiently bound with the hydroxyl sites at levels of the starch chains, they aggregate to form a V-type complex, whose position will determine the spatial distribution of the pores in the later stage. The present disclosure emphasizes that the precipitate is dried in a drying oven in a convective open environment at 35-75° C., which can provide the interfacial tension formed by ethanol evaporation and moisture migration, thereby stretching the amylose and amylopectin of different chain lengths in the system to form “perfect circular” pores. When the amylose content increases from 0% to 80%, the starch pore size gradually decreases. When amylose accounts for 50 to 80% of the compound starch, porous starch with pore sizes in the range of 1 to 1000 nm is obtained that is most suitable for encapsulating probiotics.

Further, in step (1), the plant source of purified amylose and purified amylopectin is any one of corn, potato, wheat, cassava, sweet potato, and rice.

Further, in the step (1), the concentration of the compound starch suspension is in a range of 2.5 to 15 wt %.

Further, in the step (1), the ultrasonic treatment conditions are: the ultrasonic method being one of probe ultrasonic or water bath ultrasonic, the ultrasonic power being in a range of 20 to 60 W/ml, the ultrasonic frequency being in a range of 20 to 60 kHz, the ultrasonic time being in a range of 10 to 30 min, and the temperature of the ultrasonic treated solution being in a range of 60 to 100° C.

Further, in the step (1), the alcohol precipitation includes dripping absolute ethanol into the ultrasonic treated solution at a constant rate of 5 to 25 ml/min until a volume ratio of the ethanol to the ultrasonic treated solution is in a range of 1 to 3:1 without stirring, while maintaining a temperature of the solution above 50° C.

Further, in the step (1), the centrifugation conditions are: the centrifugal force being in a range of 3500 to 6500×g, and the centrifugation time being 10 minutes.

Furthermore, the present disclosure uses the above-mentioned porous starch with a specific pore size to load probiotics, optimizing the loading process of probiotics, improving the retention rate of probiotics in complex environments, and ensuring the biological functions of probiotics. Specifically, in the present disclosure, the porous starch with pore sizes within 1 μm is sterilized and then, dispersed in a culture medium solution, to which probiotics are added to obtain a mixed solution. In the mixed solution, the concentration of starch is in a range of 0.5 to 20 wt %, the ratio of the porous starch mass to the viable count is 1 g: 1011 CFU/mL. The mixed solution is slightly gelatinized and shaken at 30-40° C. for 0.5-2 h, centrifuged, and freeze-dried to obtain starch loaded with probiotics.

Preferably, the pore size of the porous starch has a narrow distribution. The narrow distribution means that, the pores within a specified pore size range account for more than 90%. For example, small pores with pore sizes in the range of 500 nm to 1000 nm account for more than 90% of the total, or small pores with pore sizes in the range of 500 nm to 1000 nm account for more than 90% of the total.

Further, the culture medium solution is MRS broth.

Further, the starch is sterilized by ultraviolet light.

Further, the centrifugation speed is in a range of 3000 to 6500 rpm, and the centrifugation time is 10 minutes.

In some embodiments of the present disclosure, after sterilizing the porous starch by ultraviolet light, MRS broth is used as the dissolution medium, and after homogenization, a uniform porous starch suspension is formed, and a probiotic culture is added in proportion. The mixed solution is slightly gelatinized and shaken to obtain porous starch granules loaded with probiotics. Specifically, the porous starch is sterilized by ultraviolet light for 10-30 minutes, and dissolved and suspended in sterile MRS broth. The homogenization speed is in a range of 5000-10000 rpm, and the homogenization time is in a range of 1-5 minutes. The probiotic culture is added in proportion until the ratio of the starch mass to the viable count is 1 g: 1011 CFU/mL. The concentration of the porous starch suspension is in a range of 0.5 to 20 wt %. The mixed solution is slightly gelatinized at 30 to 40° C. for 0.5 to 2 h, centrifuged to take the precipitate, and the precipitate is freeze-dried for 4 to 8 h to obtain porous starch granules with probiotics encapsulated therein.

In the present disclosure, the sterilized porous starch is homogeneously suspended in the MRS solution, and after quantitatively introducing the probiotic culture, the nanoscale pores of the porous starch absorb the probiotics. The slightly gelatinized condition promotes the structural change of the porous starch, and the probiotics are encapsulated in the porous starch. The starch shell improves the retention rate of probiotics in complex environments and provides effective protection for probiotics.

The benefits are as follows:
1. The present disclosure finds the negative correlation between amylose-amylopectin ratio and pore size, and seek reasonable technological means to prepare PS with controllable pore size and morphology based on the negative correlation.
2. The present disclosure proposes a green, environmentally friendly, simple and efficient physical processing process for preparing PS. The product is safe and edible, and is a good material for adsorbing and embedding bioactive substances of different molecular weights. The encapsulated probiotics here have a retention rate above 15% under a freeze-drying condition at −50° C., and a retention rate above 95% under a heat treatment condition at 45° C.

### BRIEF DESCRIPTION OF DRAWINGS

FIG. 1 is a process flow chart for preparing PS with controllable multi-level pore size and morphology in the present disclosure.

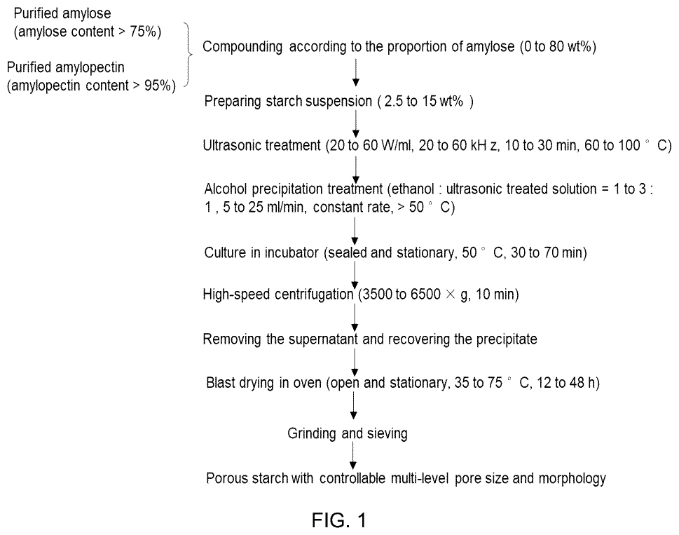

FIG. 2 shows scanning electron microscope (SEM) microscopic images of the PS with controllable multi-level pore size and morphology prepared in Example 1 of the present disclosure, which are used to directly prove the pore sizes in levels of the PS prepared in the present disclosure.

FIG. 3 shows pore size distributions of the PS with controllable multi-level pore size and morphology prepared in Example 1 of the present disclosure and the respective mercury intrusion/extrusion curves, which are used to directly prove the content distribution of the pores in levels of the PS prepared in the present disclosure.

FIG. 4 shows scanning electron microscope (SEM) microscopic images of the PS prepared by vacuum freeze-drying in Comparative Example 1 of the present disclosure, which are used to directly prove the influence of the ultrasonic treatment process in the preparation method of the present disclosure on the morphology of the pores.

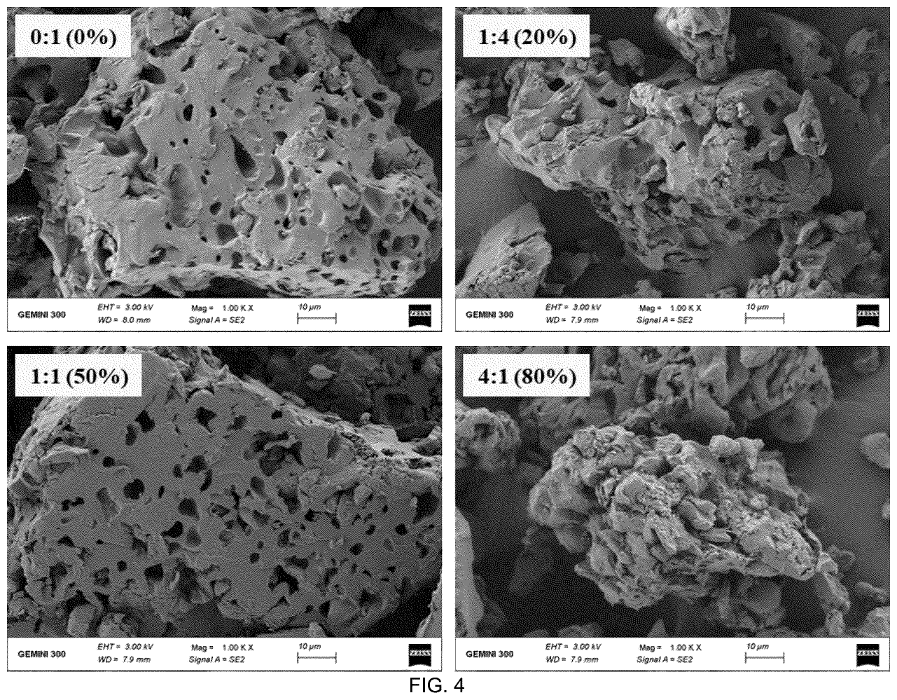

FIG. 5 shows scanning electron microscope (SEM) microscopic images of the PS prepared by vacuum freeze-drying in Comparative Example 2 of the present disclosure, which are used to directly prove the influence of the blast drying process in the preparation method of the present disclosure on the morphology of the pores.

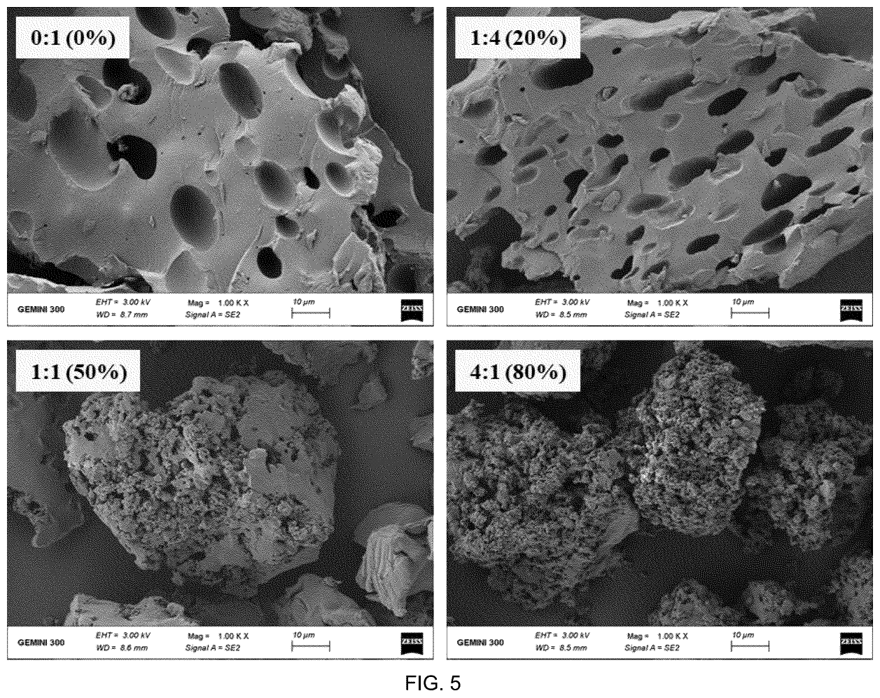

FIG. 6 shows scanning electron microscope (SEM) microscopic images of the porous starch loaded with probiotics in Examples 3 and 4, which are used to directly prove the existence of the probiotics prepared in the present disclosure and the protective effect.

FIG. 7 shows X-ray photoelectron spectroscopy (XPS) data images of the porous starch loaded with probiotics prepared in Examples 3 and 4, which are used to directly prove the existence of the probiotics.

FIG. 8 shows the scanning electron microscope (SEM) microscopic images and X-ray photoelectron spectroscopy (XPS) data images in Comparative Examples 3 and 4, which are used to prove the protective effect of porous starch on the retention rate of probiotics.

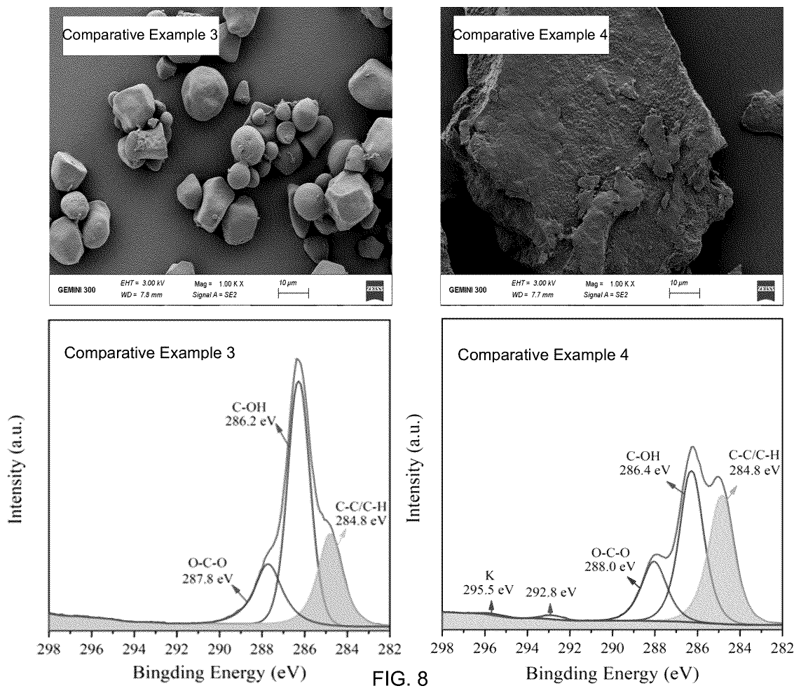

### DESCRIPTION OF EMBODIMENTS

In order to further explain the technical means and effects of the present disclosure for the objects of the present disclosure, the specific implementations, structures, features and effects of the present disclosure are described in detail below with reference to the accompanying drawings and preferred examples.

The present disclosure will be further illustrated by the following examples. The following examples are for illustrative purposes and are not intended to limit the scope of the present disclosure.

### EXAMPLE 1

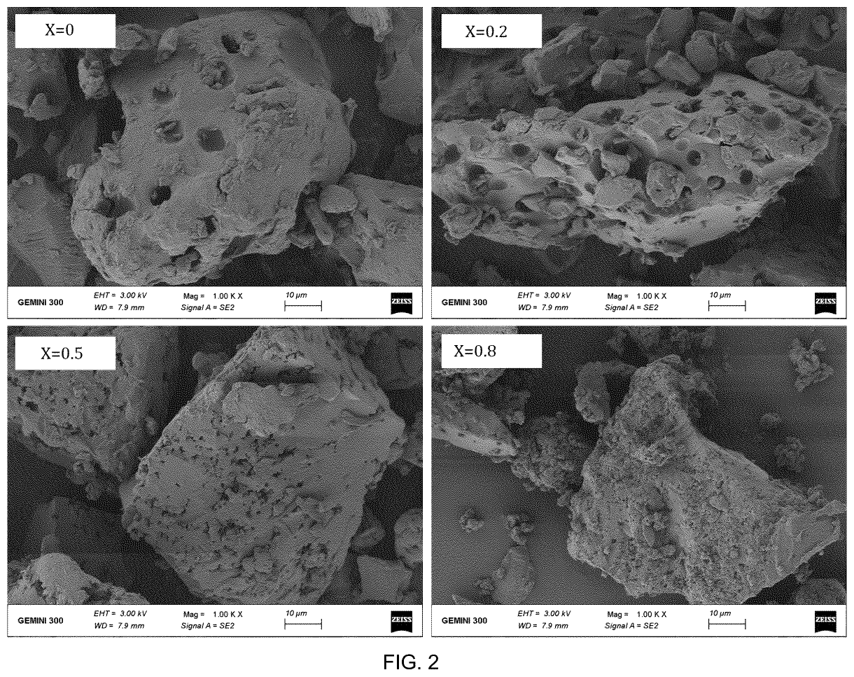

A method for preparing PS with controllable multi-level pore size and morphology includes the following steps:
(1) Preparation of compound starch suspensions: compounding corn-derived amylose and amylopectin with the proportions of amylose being 0, 20%, 50% and 80% respectively, and then preparing 2.5 wt % starch suspensions by mixing them with ionized water respectively;
(2) Ultrasonic treatment: in a probe ultrasonic working mode, inserting the probe deep into the center of the upper ⅓ of each liquid below the liquid surface, wherein the ultrasonic power is 50 W/ml, the frequency is 50 kHz, and the time is 10 min, and performing ultrasonic gelatinization while maintaining 90° C. water bath heating;
(3) Alcohol precipitation treatment: adding absolute ethanol dropwise to each ultrasonic treated solution prepared in step (2) at a constant rate of 5 ml/min until the volume ratio of ethanol to ultrasonic treated solution is 2.5:1 without stirring, while maintaining the solution temperature at least 50° C.;
(4) Centrifugation after constant temperature cultivation: sealing each mixed solution prepared in step (3) for stationary culture for 30 minutes at 50° C., and centrifuging at a centrifugal force of 3500×g for 10 minutes and then removing the supernatant to obtain the precipitate;
(5) Blast drying: placing each precipitate obtained in step (4) in a hot air drying oven with air blast function at 35° C., and drying it for 48 hours in the open oven to obtain the PS with controllable multi-level pore size and morphology;
(6) Observation of porous morphology: spreading a small amount of starch granules that have been sieved through a sieve with pore size of 75 μm evenly on a conductive adhesive and coating with gold, and observing the morphology with a scanning electron microscope (SEM) under an accelerating voltage of 3 KV at a magnification of ×10,000. As shown in FIG. 2, it can be seen that the amylose in different proportions in this example all result in porous starch with circular pores.
(7) Pore size distribution detection: using a mercury porosimeter (MIP) to apply external pressure to intrude non-wetted mercury into the PS sample, and detecting micron-scale voids through an intrusion-extrusion cycle; recording the pressure and mercury intrusion amount, and deducing the pore structure parameters (such as pore radius, pore volume, pore surface area, and pore size distribution) from the experimental intrusive mercury curve. Assuming that the pores are cylindrical pores, the relationship between pore radius and pressure can be represented by the Washburn equation as follows:

$$P = \frac{2 γcosθ}{r}$$

wherein P is the mercury intrusion pressure (MPa), r is the pore radius (μm) when mercury enters under the pressure P, θ is the contact angle) (130°), and γ is the interfacial tension of mercury (0.485 J/m²).

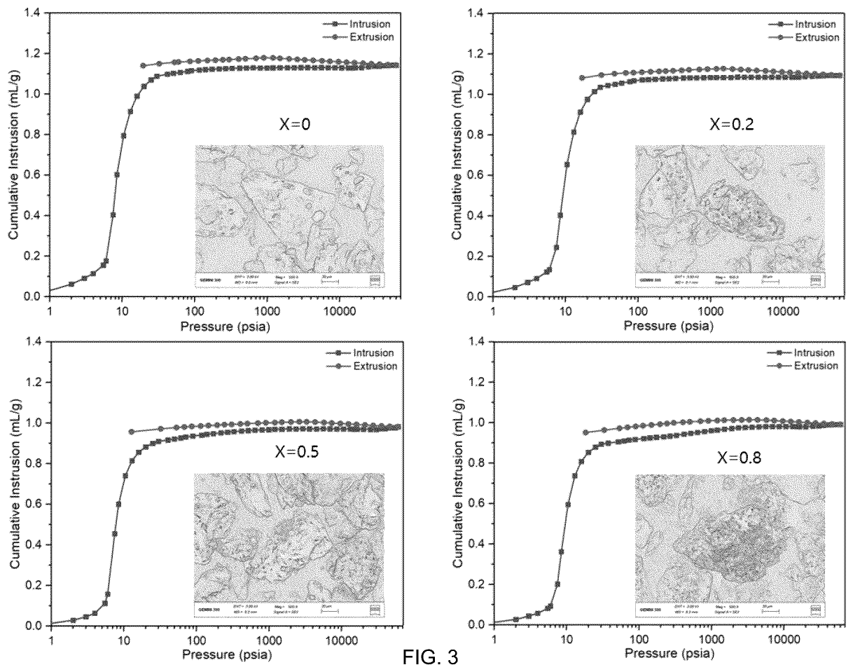

FIG. 3 shows the mercury intrusion/extrusion curve. It can be seen that as the proportion of amylose increases, the pore size of the prepared porous starch becomes smaller. Amylopectin generally has a short chain length and is prone to agglomeration, which is not easy to change during concerted reaction, so the resulting pore structure is larger. On the contrary, amylose is relatively rigid, and the starch chain is easily broken by ultrasonic physical fields, resulting in more pores. Therefore, the pore size distribution of porous starch can be controlled by compounding amylose/amylopectin.

### EXAMPLE 2 (50%≤X<80%)

A method for preparing PS with controllable multi-level pore size and morphology includes the following steps:
(1) Preparation of compound starch suspensions: compounding corn-derived amylose and amylopectin with the proportions of amylose being 50%, 60%, 70% and 79% respectively, and then preparing 10 wt % starch suspensions by mixing them with ionized water respectively;
(2) Ultrasonic treatment: in a probe ultrasonic working mode, inserting the probe deep into the center of the upper ⅓ of each liquid below the liquid surface, wherein the ultrasonic power is 20 W/ml, the frequency is 20 kHz, and the time is 25 min, and performing ultrasonic gelatinization while maintaining 60° C. water bath heating;
(3) Alcohol precipitation treatment: adding absolute ethanol dropwise to each ultrasonic treated solution prepared in step (2) at a constant rate of 20 ml/min until the volume ratio of ethanol to ultrasonic treated solution is 1:1 without stirring, while maintaining the solution temperature at least 50° C.;
(4) Centrifugation after constant temperature cultivation: sealing each mixed solution prepared in step (3) for stationary culture for 60 minutes at 50° C., and centrifuging at a centrifugal force of 6500×g for 10 minutes and then removing the supernatant to obtain the precipitate;
(5) Blast drying: placing the precipitate obtained in step (4) in a hot air drying oven with air blast function at 65° C., and drying it for 12 hours in the open oven to obtain the PS with controllable multi-level pore size and morphology;
(6) Observation of porous morphology: spreading a small amount of starch granules that have been sieved through a sieve with pore size of 75 μm evenly on a conductive adhesive and coating with gold, and observing the morphology with a scanning electron microscope (SEM) under an accelerating voltage of 3 KV at a magnification of ×10,000. It can be seen that the starch surface has relatively dense circular pores;
(7) Pore size distribution detection: using a mercury porosimeter (MIP) to apply external pressure to intrude non-wetted mercury into the PS sample, and detecting micron-scale voids through an intrusion-extrusion cycle; recording the pressure and mercury intrusion amount, and deducing the pore structure parameters (such as pore radius, pore volume, pore surface area, and pore size distribution) from the experimental intrusive mercury curve. Assuming that the pores are cylindrical pores, the relationship between pore radius and pressure can be represented by the Washburn equation as follows:

$$P = \frac{2 γcosθ}{r}$$

wherein P is the mercury intrusion pressure (MPa), r is the pore radius (μm) when mercury enters under the pressure P, θ is the contact angle) (130°), and γ is the interfacial tension of mercury (0.485 J/m²).

According to the mercury intrusion test, when the proportion of amylose is 50%, the proportion of small pores in the range of 1≤Y<1000 nm reaches 93.96%. When the proportion of amylose is 60%, the proportion of small pores in the range of 1≤Y<1000 nm reaches 92.33%. When the proportion of amylose is 70%, the proportion of small pores in the range of 1≤Y<1000 nm reaches 91.63%. When the proportion of amylose is 79%, the proportion of small pores in the range of 1≤Y<1000 nm reaches 92.51%. It can be seen that when the proportion of amylose is in the range of 50%≤X<80%, the proportion of small pores in the range of 1≤Y<1000 nm reaches over 90%.

### EXAMPLE 3

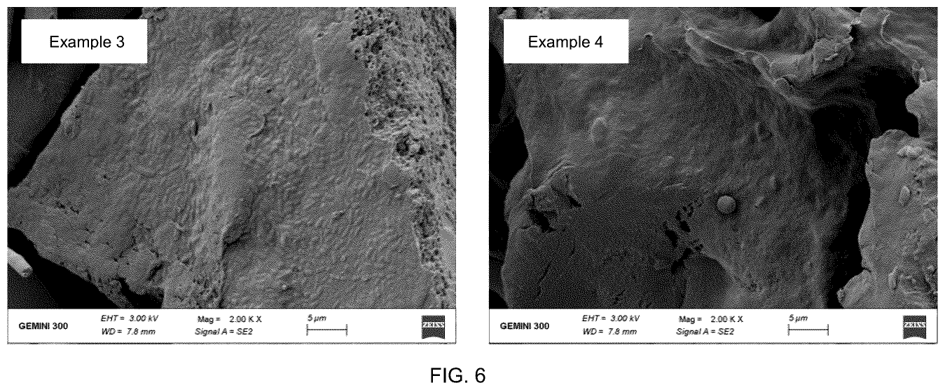

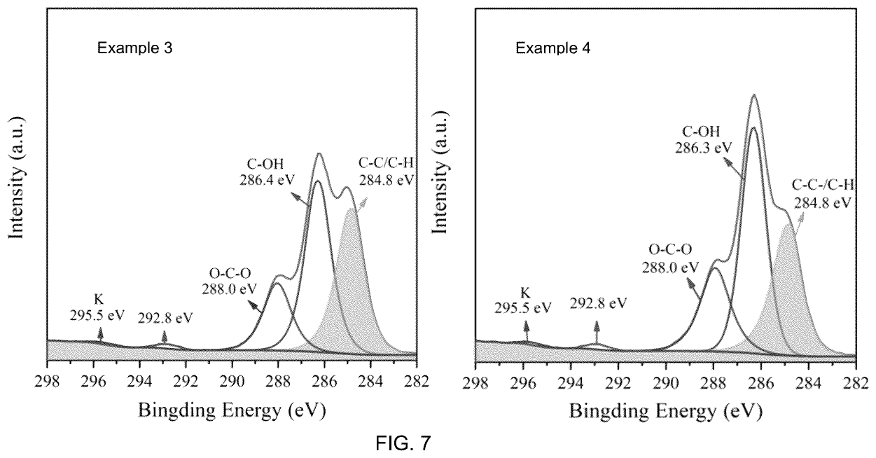

An encapsulation method with porous starch that effectively protects probiotics includes the following steps:
(1) Preparation of nanoscale porous starch: performing ultrasonic treatment on the compound starch suspension, and then performing alcohol precipitation on the ultrasonic treated solution; after performing concentration cultivation on the ultrasonic treated solution precipitated by alcohol, centrifuging and drying the precipitate at 50° C., wherein the amylose mass in the starch suspension accounts for 80% of the total mass of amylose and amylopectin;
(2) It is measured that the pore sizes of the pores of the porous starch prepared in step 1 are mainly distributed within the range of 1 to 500 nm, wherein the small pores in the range of 15 to 30 μm account for more than 90% of all of the pores; suspending the porous starch after ultraviolet sterilization in MRS broth, and homogenizing at 5000 rpm for 1 min to prepare a 10 wt % porous starch suspension;
(3) Preparation of porous starch-probiotic complex: adding probiotic culture in proportion, wherein the ratio of starch mass to viable count is 1 g: 1011 CFU/mL, and culturing in a slightly gelatinized condition at 37° C. for 1 hour;
(4) Centrifugation after the reaction: centrifuging at 5000 rpm for 10 minutes, carefully removing the supernatant, and freeze-drying the precipitate for 48 hours;
(5) Observation of probiotic morphology: spreading a small amount of starch granules that have been sieved through a sieve with pore size of 75 μm evenly on a conductive adhesive and coating with gold, and observing the morphology with a scanning electron microscope (SEM, FIG. 6) under an accelerating voltage of 3 KV at a magnification of ×10,000. It can be seen that probiotics are mostly encased in porous starch in a semi-embedded or completely embedded form;
(6) X-ray photoelectron spectroscopy (XPS, FIG. 7): determining the elemental surface composition of unencapsulated and encapsulated Lactobacillus plantarum; when the pressure is less than 2.0×10⁻⁷ mbar (12 kV, 6 mA), pressing freeze-dried bacterial powder (20-30 mg) into a small plate and placing it in the XPS chamber; calculating the binding energy at the C1s binding energy peak at 284.8 eV; performing narrow scan within the binding energy range of 20 eV to determine the chemical functionality of O1s; calculating the peak intensity from the area at each peak to obtain the elemental surface concentration ratio of nitrogen and oxygen to carbon;
(7) Determination of cold and heat resistance of probiotics: in order to evaluate the protective effect of porous starch encapsulation on probiotics, performing determination after freeze-drying (−48° C.) and heat treatment at 30° C. and 45° C. for 15 min; immersing 100 mg sample into 1 mL PBS, heating and culturing in water for 15 min, and then cooling to room temperature in an ice-water bath; assessing viable cells surviving after heat treatment by agar plate assay.

The porous starch prepared in this example is loaded with probiotics. The retention rate of the probiotics after freeze-drying is 15%, and the retention rate after heat treatment is 95%. Further, the XPS results show that with the addition of the probiotics, the nitrogen content significantly increases. This method can successfully prepare porous starch granules loaded with probiotics.

### EXAMPLE 4

Probiotics are encapsulated with the porous starch prepared in Example 2 with the proportion of amylose being 50%. The steps are as follows:
(1) Suspending the porous starch after ultraviolet sterilization in MRS broth, and homogenizing at 5000 rpm for 1 min to prepare a 10 wt % porous starch suspension;
(2) Preparation of porous starch-probiotic complex: adding probiotic culture in proportion, wherein the ratio of starch mass to viable count is 1 g: 1011 CFU/mL, and culturing in a slightly gelatinized condition at 37° C. for 1 hour;
(3) Centrifugation after the reaction: centrifuging at 5000 rpm for 10 minutes, carefully removing the supernatant, and freeze-drying the precipitate for 48 hours;
(4) Observation of probiotic morphology: spreading a small amount of starch granules that have been sieved through a sieve with pore size of 75 μm evenly on a conductive adhesive and coating with gold, and observing the morphology with a scanning electron microscope (SEM, FIG. 6) under an accelerating voltage of 3 KV at a magnification of ×10,000. It can be seen that probiotics are mostly encased in porous starch in a semi-embedded or completely embedded form;
(5) X-ray photoelectron spectroscopy (XPS, FIG. 7): determining the elemental surface composition of unencapsulated and encapsulated Lactobacillus plantarum; when the pressure is less than 2.0×10⁻⁷ mbar (12 kV, 6 mA), pressing freeze-dried bacterial powder (20-30 mg) into a small plate and placing it in the XPS chamber; calculating the binding energy at the C1s binding energy peak at 284.8 eV; performing narrow scan within the binding energy range of 20 eV to determine the chemical functionality of O1s; calculating the peak intensity from the area at each peak to obtain the elemental surface concentration ratio of nitrogen and oxygen to carbon;
(6) Determination of cold and heat resistance of probiotics: in order to evaluate the protective effect of porous starch encapsulation on probiotics, performing determination after freeze-drying (−48° C.) and heat treatment at 30° C. and 45° C. for 15 min; assessing viable cells surviving after heat treatment by agar plate assay.

The porous starch prepared in this example is loaded with probiotics. The retention rate of the probiotics after freeze-drying is 18%, and the retention rate after heat treatment is 99%. Further, the XPS results show that with the addition of the probiotics, the nitrogen content significantly increases. This method can successfully prepare porous starch granules loaded with probiotics.

### Comparative Example 1

A method for preparing PS with controllable multi-level pore size and morphology includes the following steps:
(1) Preparation of compound starch suspensions: compounding corn-derived amylose and amylopectin in four ratio of 0:1, 1:4, 1:1, and 4:1 respectively, that is, the proportions of amylose being 0, 20%, 50% and 80% respectively, and then preparing 5 wt % starch suspensions by mixing them with ionized water respectively;
(2) In order to prove the influence of ultrasonic treatment on the formation of pore size here, this comparative example does not include an ultrasonic treatment step, that is, the starch suspension prepared in step (1) is directly used for alcohol precipitation treatment;
(3) Alcohol precipitation treatment: adding absolute ethanol dropwise to each ultrasonic treated solution prepared in step (1) at a constant rate of 20 ml/min until the volume ratio of ethanol to ultrasonic treated solution is 1.5:1 without stirring, while maintaining the solution temperature at least 50° C.;
(4) Centrifugation after constant temperature cultivation: sealing the four mixed solutions prepared in step (3) for stationary culture for 30 minutes at 50° C., and centrifuging at a centrifugal force of 3500 to 6500 x g for 10 minutes and then removing the supernatant to obtain four types of precipitates respectively;
(5) Blast drying: placing the precipitates obtained in step (4) in a hot air drying oven with air blast function at 50° C., and drying them for 24 hours in the open oven to obtain four types of PS prepared without ultrasonic treatment;
(6) Observation of porous morphology: spreading a small amount of starch granules that have been sieved through a sieve with pore size of 75 μm evenly on a conductive adhesive and coating with gold, and observing the morphology with a scanning electron microscope (SEM) under an accelerating voltage of 3 KV at a magnification of ×10,000.

The PS prepared in this comparative example has uniform pore size, without correlation with the ratio of amylose to amylopectin, that is, PS with controllable pore size could not be successfully prepared.

### Comparative Example 2

(1) Preparation of compound starch suspensions: compounding corn-derived amylose and amylopectin in four ratio of 0:1, 1:4, 1:1, and 4:1 respectively, that is, the proportions of amylose being 0, 20%, 50% and 80% respectively, and then preparing 5 wt % starch suspensions by mixing them with ionized water respectively;
(2) Ultrasonic treatment: in a probe ultrasonic working mode, inserting the probe deep into the center of the upper ⅓ of each liquid below the liquid surface, wherein the ultrasonic power is 20 W/ml, the frequency is 20 kHz, and the time is 20 min, and performing ultrasonic gelatinization while maintaining 70° C. water bath heating;
(3) Alcohol precipitation treatment: adding absolute ethanol dropwise to each ultrasonic treated solution prepared in step (2) at a constant rate of 20 ml/min until the volume ratio of ethanol to ultrasonic treated solution is 1.5:1 without stirring, while maintaining the solution temperature at least 50° C.;
(4) Centrifugation after constant temperature cultivation: sealing the four mixed solutions prepared in step (3) for stationary culture for 30 minutes at 50° C., and centrifuging at a centrifugal force of 3500 to 6500×g for 10 minutes and then removing the supernatant to obtain four types of precipitates respectively;
(5) In order to prove the influence of the blast drying process on the pore morphology here, this comparative example uses vacuum freeze drying instead of the blast drying step. The precipitates obtained in step (4) are vacuum freeze-dried at 50° C. for 48 hours to obtain four types of freeze-dried PS;
(6) Observation of porous morphology: spreading a small amount of starch granules that have been sieved through a sieve with pore size of 75 μm evenly on a conductive adhesive and coating with gold, and observing the morphology with a scanning electron microscope (SEM) under an accelerating voltage of 3 KV at a magnification of ×10,000.

The pore size of the PS prepared in this comparative example has a weak correlation with the ratio of amylose to amylopectin, but the pore morphology assumes an elliptical due to the gravity of dripped ethanol, that is, PS with controllable morphology could not be successfully prepared.

### Comparative Example 3 Using Common Corn Starch as a Probiotic Carrier

(1) Preparation of common starch suspension: dissolving and suspending common corn starch after ultraviolet sterilization in MRS broth, and homogenizing at 5000 rpm for 1 min to prepare a 10 wt % starch suspension;
(2) Preparation of starch-probiotic complex: adding probiotic culture in proportion, wherein the ratio of starch mass to viable count is 1 g: 1011 CFU/mL, and culturing in a slightly gelatinized condition at 37° C. for 1 hour;
(3) Centrifugation after the reaction: centrifuging at 5000 rpm for 10 minutes, carefully removing the supernatant, and freeze-drying the precipitate;
(4) Observation of probiotic morphology: spreading a small amount of starch granules that have been sieved through a sieve with pore size of 75 μm evenly on a conductive adhesive and coating with gold, and observing the morphology with a scanning electron microscope (SEM) under an accelerating voltage of 3 KV at a magnification of ×10,000. It can be seen that the surface of the starch has relatively dispersed and sporadic probiotics.
(5) X-ray photoelectron spectroscopy (XPS): determining the elemental surface composition of unencapsulated and encapsulated Lactobacillus plantarum; when the pressure is less than 2.0×10⁻⁷ mbar (12 kV, 6 mA), pressing freeze-dried bacterial powder (20-30 mg) into a small plate and placing it in the XPS chamber; calculating the binding energy at the C1s binding energy peak at 284.8 eV; performing narrow scan within the binding energy range of 20 eV to determine the chemical functionality of O1s; calculating the peak intensity from the area at each peak to obtain the elemental surface concentration ratio of nitrogen and oxygen to carbon;
(6) Determination of cold and heat resistance of probiotics: in order to evaluate the protective effect of porous starch encapsulation on probiotics, performing determination after freeze-drying (−48° C.) and heat treatment at 30° C. and 45° C. for 15 min; assessing viable cells surviving after heat treatment by agar plate assay.

The common starch prepared in this comparative example is loaded with probiotics. The retention rate of the probiotics after freeze-drying is 0.24%, and the retention rate after heat treatment is 6.2%. Further, the XPS results show that with the addition of the probiotics, the nitrogen content significantly increases. The retention rate of the loaded probiotics in this comparative example is significantly lower than that in the porous starch with nanoscale pores.

### Comparative Example 4 Using Porous Starch with Microscale Pores as a Probiotic Carrier

(1) Preparation of microscale porous starch: performing ultrasonic treatment on the compound starch suspension, and then performing alcohol precipitation on the ultrasonic treated solution; after performing concentration cultivation on the ultrasonic treated solution precipitated by alcohol, centrifuging and drying the precipitate at 50° C., wherein the amylose mass in the starch suspension accounts for 10% of the total mass of amylose and amylopectin;
(2) It is measured that the pore sizes of the pores of the porous starch prepared in step 1 are mainly distributed within the range of 15 to 30 um, wherein the small pores in the range of 15 to 30 um account for more than 90% of all of the pores; suspending the porous starch after ultraviolet sterilization in MRS broth, and homogenizing at 5000 rpm for 1 min to prepare a 10 wt % porous starch suspension;
(3) Preparation of starch-probiotic complex: adding probiotic culture in proportion, wherein the ratio of starch mass to viable count is 1 g: 1011 CFU/mL, and culturing in a slightly gelatinized condition at 37° C. for 1 hour;
(4) Centrifugation after the reaction: centrifuging at 5000 rpm for 10 minutes, carefully removing the supernatant, and freeze-drying the precipitate;
(5) Observation of probiotic morphology: spreading a small amount of starch granules that have been sieved through a sieve with pore size of 75 μm evenly on a conductive adhesive and coating with gold, and observing the morphology with a scanning electron microscope (SEM) under an accelerating voltage of 3 KV at a magnification of ×10,000. It can be seen that most of the probiotics are floating on the surface of the porous starch;
(6) X-ray photoelectron spectroscopy (XPS): determining the elemental surface composition of unencapsulated and encapsulated Lactobacillus plantarum; when the pressure is less than 2.0x10⁻⁷ mbar (12 kV, 6 mA), pressing freeze-dried bacterial powder (20-30 mg) into a small plate and placing it in the XPS chamber; calculating the binding energy at the C1s binding energy peak at 284.8 eV; performing narrow scan within the binding energy range of 20 eV to determine the chemical functionality of O1s; calculating the peak intensity from the area at each peak to obtain the elemental surface concentration ratio of nitrogen and oxygen to carbon;
(7) Determination of cold and heat resistance of probiotics: in order to evaluate the protective effect of porous starch encapsulation on probiotics, performing determination after freeze-drying (−48° C.) and heat treatment at 30° C. and 45° C. for 15 min; assessing viable cells surviving after heat treatment by agar plate assay.

The porous starch prepared in this comparative example is loaded with probiotics. The retention rate of probiotics after freeze-drying is 0.06%, and the retention rate after heat treatment is 1.5%. The XPS results show that with the addition of the probiotics, the nitrogen content significantly increases. The retention rate of the loaded probiotics in this comparative example is significantly lower than that in the porous starch with nanoscale pores.

The above are only preferred examples of the present disclosure, and do not limit the present disclosure in any sense. Although the present disclosure is described above referring to preferred examples, the preferred examples are not intended to limit the present disclosure. One skilled in the art, without departing from the scope of the technical solution of the present disclosure, can make some changes or modifications to the technical contents disclosed above to obtain equivalent examples with equivalent changes. Without departing from the technical solution of the present disclosure, any simple modifications and equivalent changes made to the above examples based on the technical concept of the present disclosure still fall within the scope of the technical solution of the present disclosure.

1. A preparation method for porous starch for encapsulating probiotics, comprising:
performing ultrasonic treatment on a compound starch suspension to obtain an ultrasonic treated solution, and then performing alcohol precipitation on the ultrasonic treated solution;
performing concentration cultivation on the ultrasonic treated solution precipitated by alcohol and then centrifuging to obtain a precipitate; and
drying the precipitate at a temperature in a range of 35° C. to 75° C. to obtain porous starch with a pore size in a range of 1 to 1000 nm;
wherein a compound starch in the compound starch suspension consists of amylose and amylopectin, and the amylose accounts for 50% to 80% of the compound starch.

2. The preparation method according to claim 1, wherein a plant source of the amylose and the amylopectin is any one of corn, potato, wheat, cassava, sweet potato, and rice.

3. The preparation method according to claim 1, wherein the compound starch suspension has a concentration in a range of 2.5 to 15 wt %.

4. The preparation method according to claim 1, comprising performing the ultrasonic treatment at an ultrasonic power in a range of 20 to 60 W/ml, an ultrasonic frequency in a range of 20 to 60 KHz, an ultrasonic time in a range of 10 to 30 min, and an ultrasonic temperature in a range of 60 to 100° C.

5. The preparation method according to claim 1, wherein the alcohol precipitation comprises dripping absolute ethanol into the ultrasonic treated solution at a constant rate of 5 to 25 ml/min until a volume ratio of the ethanol to the ultrasonic treated solution is in a range of 1 to 3:1 without stirring, while maintaining a temperature of the solution above 50° C.

6. The preparation method according to claim 1, comprising performing the concentration cultivation at a sealing and stationary condition, with a cultivation temperature in a range of 50° C. to 70° C., and a cultivation time in a range of 30 to 70 min.

7. The preparation method according to claim 1, comprising performing the centrifuging at a centrifugal force in a range of 3500 to 6500×g, and a centrifugation time of 10 min.

8. The preparation method according to claim 1, wherein the drying comprises drying for 12 to 48 hours with hot air in a convective open environment.

9. An encapsulating method for probiotics using the porous starch prepared by the method of claim 1, comprising:
sterilizing the porous starch and then homogeneously dispersing the same in a culture medium solution, and adding the probiotics therein to obtain a mixed liquid; wherein in the mixed liquid, the porous starch has a concentration in a range of 0.5 to 20 wt %, and a ratio of a porous starch mass to a viable count is 1 g: 1011 CFU/mL; and
slightly gelatinizing and shaking the mixed liquid for 0.5 to 2 h at 30 to 40° C., centrifuging, and freeze-drying to obtain starch loaded with probiotics.

10. The method according to claim 9, wherein the culture medium solution is MRS broth.

11. The method according to claim 9, comprising sterilizing the porous starch by ultraviolet light.

12. The method according to claim 9, comprising performing the centrifuging at a centrifugal speed in a range of 3000 to 6500 rpm and a centrifugation time of 10 minutes.

13. The method according to claim 9, comprising after dispersing the porous starch in the culture medium solution, performing homogenization to form a uniform porous starch suspension.
